(19) United States
(12) Patent Application Publication
(10) Pub. No.: US 2026/0014301 A1
(43) Pub. Date: Jan. 15, 2026

(54) ORGANO-SELENIUM-INCORPORATED URINARY CATHETER TUBING

(71) Applicant: Texas Tech University System, Lubbock, TX (US)

(72) Inventors: Ted W. REID, Wolfforth, TX (US); Werner T.W. DE RIESE, Lubbock, TX (US); Phat L. TRAN, Lubbock, TX (US)

(73) Assignee: TEXAS TECH UNIVERSITY SYSTEM, Lubbock, TX (US)

(21) Appl. No.: 18/992,069

(22) PCT Filed: Jul. 7, 2023
(86) PCT No.: PCT/US23/69809

§ 371 Date: Jan. 7, 2025

Related U.S. Application Data
(60) Provisional application No. 63/358,893, filed on Jul. 7, 2022.

(51) Int. Cl. A61L 29/12; A61L 29/16

(52) U.S. Cl. CPC A61L 29/126; A61L 29/16; A61L 2300/102; A61L 2300/404; A61L 2420/04

(57) ABSTRACT

Disclosed is a catheter tubing having surface expression of organo-selenium compositions capable of presenting anti-microbial properties on the surface of the catheter tubing which prevent microbial growth and the formation of biofilms. The disclosed compositions and methods support a wide variety of scenarios for catheter tubing applications, as organo-selenium can be stably incorporated into catheter tubing to inhibit bacterial attachment, growth, and biofilm formation for multiple uropathogenic organisms for long-term use with minimal to no leaching.

### CROSS-REFERENCE TO RELATED APPLICATIONS

The present application is a PCT application of U.S. Provisional Application Ser. No. 63/358,893 with a filing date of Jul. 7, 2022, entitled “Organo-Selenium-Incorporated Urinary Catheter Tubing” and which is hereby incorporated herein by reference in its entirety for all purposes.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

### TECHNICAL FIELD

The present invention relates in general to the field of catheter tubing. In particular, the present invention provides for anti-microbial catheter tubing incorporated with an organ-selenium compound. The tubing in accordance with the present invention results in improved anti-microbial properties with minimal toxicity risk. The disclosed compositions and methods support a wide variety of scenarios for catheter applications as well as related products and services.

### STATEMENT OF FEDERALLY FUNDED RESEARCH

None.

### BACKGROUND OF THE DISCLOSURE

Urinary catheter use is common, with an estimated 15-25% of hospitalized patients in the United States receiving a urinary catheter during their stay [1]. Indications for urinary catheters include surgical perioperative use, acute urinary retention, urinary obstruction, and immobilized or hospice patients [2]. Catheter-associated urinary tract infections (CAUTIs) are among the most common preventable and healthcare-acquired infections. Some 70-80% of nosocomial urinary tract infections (UTI) are associated with urinary catheters, making catheter placement a significant risk factor for infection [3]. Although CAUTIs are considered preventable, there are about 450,000 cases in the United States annually with treatment-associated costs exceeding $350 million each year, which are not eligible for Medicare or Medicaid reimbursement since 2008 [4]. CAUTIs are associated with significantly increased length of hospitalization and patient morbidity [5]. Additionally, 2-4% of CAUTI cases progress to bacteremia and sepsis, causing increased mortality [6].

Different types of antimicrobial catheter coatings have been studied for their potential in preventing CAUTIs by inhibiting bacterial colonization and biofilm formation. Biofilms form when bacteria adhere to host cells or catheter surfaces, and then aggregate by forming an extracellular polymeric substance within which microorganisms can divide and grow well protected from antimicrobial agents [6]. Therefore, biofilm formation is one of the most important factors for uropathogenic organisms in developing antibiotic resistance and persistent bacteriuria. An ICU study found that most CAUTIs are caused by gram-negative bacteria resistant to common antibiotics, indicating a real need for more effective measures in preventing infections and secondary antibiotic resistance [7]. Escherichia coli, Pseudomonas aeruginosa, and Klebsiella are the most common bacterial culprits for reported nosocomial CAUTIs [8]. It is well documented that antimicrobial catheter coatings reduce the risks for CAUTIs through material-specific biocidal properties, and thus interrupting biofilm formation [1]. Antimicrobial coated catheters have demonstrated a decline in CAUTIs when compared to uncoated catheters [9, 10, 11]. A recent meta-analysis of clinical trials on antimicrobial-coated catheters with substances such as silver alloy, noble metal alloy, and nitrofurazone, revealed that coated catheters significantly reduce the incidence of CAUTIs in patients when compared to uncoated catheters [11]. However, heterogeneity in methodology, coating materials, study power, and CAUTI definition between studies has limited the adoption of antimicrobial catheters in current medical practice.

With the continuing prevalence of CAUTIs within healthcare settings, there remains a clear need in the art for further developments in anti-microbial properties of catheters.

### SUMMARY OF THE DISCLOSURE

A promising element that has been under investigation for its antimicrobial properties is selenium, which has the unusual ability to catalyze the formation of superoxide radicals. As described herein, Selenium takes an electron from sulfur compounds such as glutathione, which is found in all body tissues, and gives it to oxygen, resulting in the formation of superoxide. This radical causes oxidative stress within bacterial cells with secondary cell death [13]. These radicals have a short half-life due to dismutation and thus have no effect on the surrounding tissue. This lack of in-vivo toxicity is well documented in studies on organo-selenium coated contact lenses and hemodialysis catheters [14,15].

It is therefore an object of the present invention to improve catheter tubing compositions by incorporating anti-microbial compounds into the catheter tubing. In one aspect, a catheter is coated with an anti-microbial agent. In another aspect, an organo-selenium compound is incorporated onto the surface of a catheter tube. In another embodiment, the organo-selenium compounds present in the catheter tubing are chemically reactive and capable of eliminating microbial populations exposed to the organo-selenium compound incorporated into the catheter tubing.

It is one object of the present invention to provide for an anti-microbial catheter tube, wherein the catheter tube comprises: an outer surface; and an inner surface positioned in the interior of the outer surface, wherein: said inner surface comprises a polymer/plastic, said inner surface is operable for microbial contact with the surface, said surface comprises an organo-selenium composition incorporated into the polymer/plastic, and said organo-selenium composition is operable to generate superoxide free radical species upon contact with microbes to inhibit microbial growth on said inner surface, and said organo-selenium composition does not leach from said inner surface.

It is another object of the present invention to provide for an anti-microbial catheter tube wherein said outer surface comprises a polymer/plastic, said outer surface is operable for microbial contact with the surface, said surface comprises an organo-selenium composition incorporated into the polymer/plastic, and said organo-selenium composition is operable to generate superoxide free radical species upon contact with microbes to inhibit microbial growth on said outer surface, and said organo-selenium composition does not leach from said outer surface.

According to another aspect the invention provided for a method for inhibiting microbial growth on a catheter tube, comprising: selecting a catheter tube comprising an organo-selenium composition, wherein said organo-selenium composition diminishes or prevents microbial growth on said catheter tube.

According to another aspect, the invention comprises a method of making selenium-incorporated catheter tubing comprising the steps of dissolving a selenium biocidal composition in a plasticiser then compounding the plasticiser into a polymer and forming the polymer into a desired shape of the catheter tubing.

It is another object of the present invention to provide a method of making anti-microbial tubing capable of microbial inhibition, wherein the method comprises forming a catheter tube having a surface that comprises a organo-selenium composition incorporated into the polymer/plastic, wherein incorporated said organo-selenium composition is operable to inhibit microbial species present on the surface of said catheter tube such that microbial growth or biofilm formation is diminished or prevented, and said organo-selenium composition does not does not leach from said surface of said catheter tube.

In one aspect said organo-selenium composition is fixed within the polymer/plastic. In another aspect, said organo-selenium composition is incapable of escaping the polymer/plastic.

In another aspect said polymer/plastic is selected from a group consisting of: nylon, declon, polyvinyl chloride, Polyvinyl chloride, silicone, poly butadiene, polyacrylonitrile, methacrylates, polyactic acid, aromatic polyesters thermoplastic polyurethane resin, high density polyethylene, high pressure, low density polyethylene, polypropylene, high-impact polystyrene, polyamides, PVC plasticisers, ABS, MBS, and combinations thereof.

In another aspect, said organo-selenium composition comprises a diselenide. In another aspect, said organo-selenium composition is selected from a group consisting of: diselenide (—Se—Se—), selenol (—SeH), diselenide methacrylate, compounds capable of polymerization having a selenol, compounds capable of polymerization having a diselenide, and combinations thereof.

In another aspect said organo-selenium composition is incorporated into the polymer/plastic as a coating on one or more layers of polymer/plastic film. Additionally, said organo-selenium composition is distributed throughout said polymer/plastic.

In another aspect, said organo-selenium composition is uniformly distributed throughout said polymer/plastic.

The systems, methods and compositions are set forth in part in the description which follows, and in part will be obvious from the description, or can be learned by practice of the methods, apparatuses, and systems. The advantages of the systems, methods and compositions will be realized and attained by means of the elements and combinations particularly pointed out in the appended claims. It is to be understood that both the foregoing general description and the following detailed description are exemplary and explanatory only and are not restrictive of the systems, methods and compositions, as claimed.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 depicts a graph showing effects of a selenium polymer catheter on the growth of Escherichia coli MM294 GFP.

FIG. 2 depicts a graph showing effects of a selenium polymer catheter on the growth of Haemophilus influenza (Clinical Isolate).

FIG. 3 depicts a graph showing effects of a selenium polymer catheter on the growth of Klebsiella pneumonia (Clinical Isolate).

FIG. 4 depicts a graph showing effects of a selenium polymer catheter on the growth of Pseudomonas aeruginosa PAO1 GFP.

FIG. 5 depicts a graph showing effects of a selenium polymer catheter on the growth of Escherichia coli MM294 after selenium polymer catheter is soaked in PBS for 12 weeks at 37° C.

FIGS. 6A-6D depict CLSM microscope images of control and selenium polymer catheter tubing after 12 weeks at 37° C.

FIGS. 7A-7P depict electron microscopy visualization of inner and outer catheter surfaces in control and organo-selenium-coated catheters following 24-hour incubation with uropathogenic bacteria.

### DETAILED DESCRIPTION OF THE DISCLOSURE

While the making and using of various embodiments of the present invention are discussed in detail below, it should be appreciated that the present invention provides many applicable inventive concepts that can be embodied in a wide variety of specific contexts, goods, or services. The specific embodiments discussed herein are merely illustrative of specific ways to make and use the disclosure and do not delimit the scope of the disclosure.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The present invention will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Catheter-associated urinary tract infections (CAUTIS) are of significant medical burden in cost, morbidity, and mortality. Antimicrobial-coated catheters have shown efficacy in preventing CAUTIs. While selenium has demonstrated antimicrobial properties with minimal toxicity risk, it has not been tested in the context of urinary catheters. It is therefore one embodiment of the present invention to provide organo-selenium-incorporated urinary catheters for inhibition of in-vitro growth and biofilm formation of common uropathogenic bacteria.

Selenium has been previously demonstrate to have the ability to catalyze the formation of superoxide radicals. This is demonstrated by the following equation of the selenide anion reduction pathway resulting in generation of superoxide radical species:

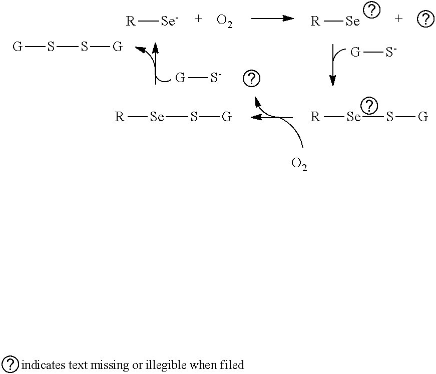

Equation of the selenide anion reduction pathway and generation of superoxide.

As outlined above, Selenium takes an electron from sulfur compounds such as glutathione, which is found in all body tissues, and gives it to oxygen, resulting in the formation of a superoxide radical. This radical causes oxidative stress within bacterial cells with secondary cell death [13]. These radicals have a short half-life due to dismutation and thus have no effect on the surrounding tissue. This lack of in-vivo toxicity is well documented in studies on organo-selenium coated contact lenses and hemodialysis catheters [14, 15].

Covalently incorporated selenium contact lenses and hemodialysis catheters have further demonstrated significant inhibition of bacterial colonization and biofilm formation on surfaces in vitro with no toxicity to animal models [14, 15]. In a recently published in-vitro study urinary catheters were inoculated with the uropathogenic Escherichia coli (UPEC) and then treated with an organic selenium which demonstrated antimicrobial activity in preventing UPEC biofilm formation when compared to the control catheters [16]. However, catheters with covalently incorporated selenium have not been tested in preventing CAUTIs.

Given the significant burden of CAUTIs for patients in cost, morbidity, and mortality, there is a real need for new measures to prevent CAUTIs. Novel organoselenium-incorporated urinary catheters set forth in the present invention are shown to prevent bacterial colonization and biofilm formation. In a further embodiment the organoselenium-incorporated catheters provide for inhibition of organoselenium-incorporated catheters on the in-vitro growth of E. coli, H. influenzae, P. aeruginosa, and K. pneumoniae and on their biofilm formation.

In one embodiment, organo-selenium-incorporated catheters demonstrated total reduction (100%) of in-vitro bacterial growth and biofilm formation compared to control, including tubing after soaking in phosphate-buffered saline for 12-weeks. Fluorescence and electron microscopy studies showed significant growth inhibition by experimental catheters compared to control. The results demonstrate that organo-selenium can be stably incorporated into catheter tubing to inhibit bacterial attachment, growth, and biofilm formation for multiple uropathogenic organisms. Furthermore, long-term soaking of organo-selenium tubing material did not show any decline in preventing bacterial growth and biofilm formation. Organo-selenium-incorporated catheters therefore will help prevent catheter-associated urinary tract infections.

### Example

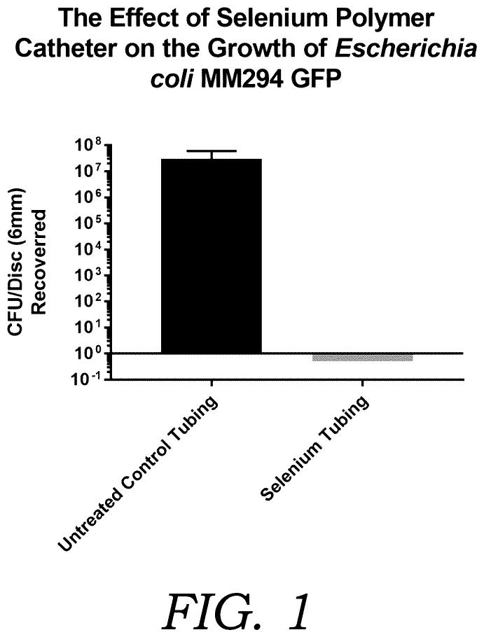

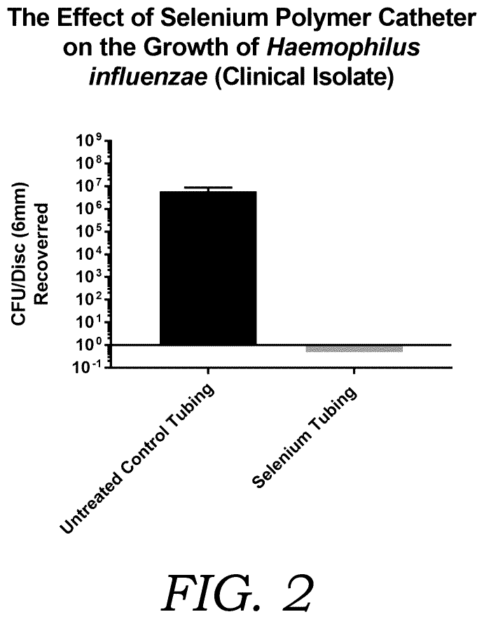

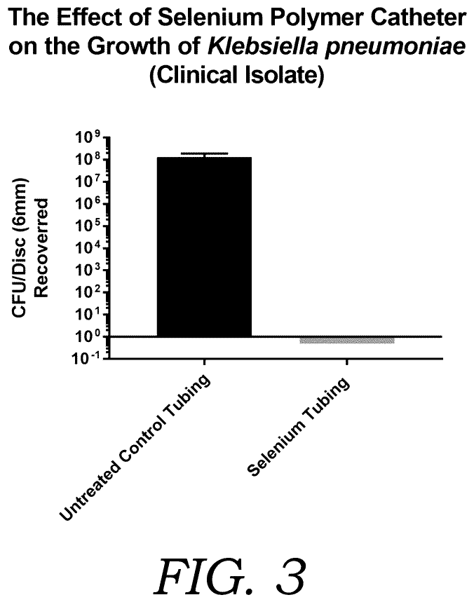

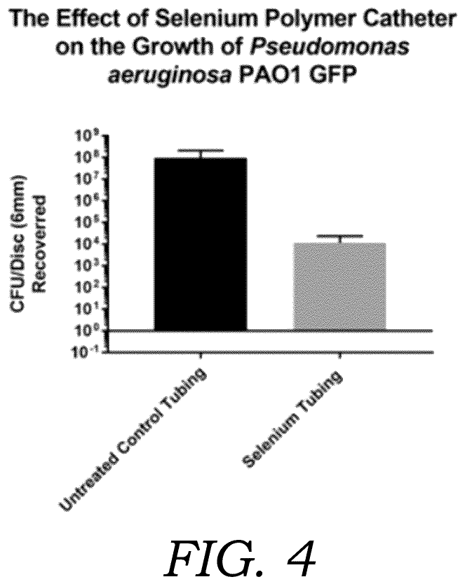

The following exemplary embodiment presents urinary catheters with 1% organo-selenium-incorporated tubing and standard (uncoated) catheters, which were in-vitro incubated with E. coli, H. influenzae, and K. pneumoniae and evaluated for growth by counting colony forming units and confocal laser microscopy (see FIGS. 1-4). Additionally, 24-hour growth of E. coli, K. pneumoniae, P. aeruginosa, and a combination of all three species were assessed by scanning electron microscopy. Catheter tubing was also tested with E. coli after long-term soaking in phosphate-buffered saline for 12-weeks at 37° C.

In an exemplary embodiment, diselenide-dimethacrylate was reactively grafted with thermoplastic polyurethane resin pellets at 235° C. The seleno-methacrylate used was added to the pellet to produce a mixture that contained 1% w/w elemental selenium. This was then thoroughly mixed to coat the pellets. The coated pellets were extruded with a bench-top injection mold. This process actually polymerizes the selenium methacrylate into the polyurethane of the tubing, forming a co-polymer. The tubing was then tested for chemical and microbiology properties. After this material was validated, the mixture was scaled-up by adding the selenium compound by auger or injection to the heating zones. This procedure was used to prepare master batches at a kilogram scale. The method can be transferred to a commercial injection molding facility to produce the material at scale and volume. The selenium tubing produced by this method was clear, and the physical properties were the same as standard non-selenium tubing. It was also found that both aged and non-aged tubing demonstrated the same physical properties.

### Bacterial Strains, Media, and Growth Conditions.

The strains of Escherichia coli GFP MM294 GFP and Pseudomonas aeruginosa PAO1 GFP, which constitutively expresses green fluorescent protein (GFP) from plasmids pMRP9-1 [17], were used in the study. We also utilized Haemophilus influenzae clinical isolate (CI) and Klebsiella pneumoniae clinical isolate. The E. coli MM294 GFP, P. aeruginosa PAO1 GFP, and K. pneumoniae clinical isolate strains were routinely grown in Luria-Bertani (LB) broth with shaking (250 rpm) or on LB Agar plates at 37° C. The H. influenzae clinical isolate was routinely grown in Brain Heart Infusion (BHI) broth with shaking (250 rpm) or on BHI Agar plates at 37° C. To maintain pMRP9-1 in E. coli MM294 GFP and P. aeruginosa PAO1 GFP, carbenicillin was incorporated into the growth medium at a concentration of 300 μg/ml. Overnight culture of the bacteria was diluted with phosphate-buffered saline (PBS), pH 7.4 to an absorbance of 0.1 at 600 nm, of which matches the 0.5 McFarland standard [24]. At this absorbance, there were approximately 1×10⁷ CFU/mL. The diluted bacterial suspension was serially diluted 10-fold in PBS (pH=7.4), and 10-μL aliquot of the negative 2 dilution was used to inoculate into each well in the Quantitative Analysis of the Biofilms (CFU/segment) section.

### Quantitative Analysis of the Biofilms (Colony Forming Unit/Segment).

The biofilm assays for the present exemplary embodiment were done as previously described [15]. Pieces (1-cm) of control and selenium catheter tubing were placed in each well of a 24-well microtiter plate (Catalog no. CC7672-7524, CytoOne, USA Scientific, Ocala, FL, USA) and incubated in media with 102-103 colony forming unit (CFU) E. coli MM294 GFP, P. aeruginosa PAO1 GFP, K. pneumoniae CI, and/or H. influenzae CI in the presence of 150 μM reduced glutathione for 24 hours. After incubation period, bacterial biofilms were quantified by determination of the CFU per tubing segment. The tubing segment was also examined by Confocal Laser Scanning Microscopy (CLSM) and/or Scanning Electron Microscopy (SEM). In the CFU assay, each segment was removed carefully from the well, rinsed gently with sterile distilled H2O to remove loosely attached bacteria, and placed into a microcentrifuge tube containing 1 ml of phosphate-buffered saline (PBS). The tubes were sonicated for 10 min to loosen the cells within the biofilm and then vigorously vortexed three times for 1 min to detach the cells. The cells were then serially diluted PBS, and 10 μl aliquots of each dilution were spotted onto LB or BHI agar plates. The plates were incubated at 37° C. for 24 h, and the CFU were determined. Using the following formula, the CFU per segment was determined: CFU×dilution factor×100. Each sample was done in triplicate.

### Scanning Electron Microscopy (SEM) Analysis.

Biofilms formed by E. coli MM294 GFP, H. influenzae CI, K. pneumoniae CI, and/or P. aeruginosa PAO1 GFP on catheter tubing were prepared for SEM by standard techniques and the experiment was performed as previously described [23]. After 48 h of incubation at 37° C., the samples were freeze-dried overnight. Any attached media was gently wiped using Kimwipes and shaken off from the samples. After mounting the samples, a thin layer of Pt was coated onto the samples to improve conductivity. The images were taken by Hitachi S/N-4300 under Environmental mode with 20 kV accelerating voltage and 300 Pa air pressure. Five fields of view at 5000× magnification was taken at randomly chosen areas from the optic surface of each sample. A biofilm-positive field was defined as being occupied by biofilm over at least half of the visible area. If no biofilm or only scant numbers of cells are seen, examination was carried out at 5000×. Experiments were done in triplicate.

### Confocal Laser Scanning Microscopy (CLSM) Analysis.

The biofilms were visualized using CLSM. The biofilms were developed as described above. After 24 h of incubation at 37° C., the pieces were gently rinsed to remove loosely attached bacteria. Visualization of the E. coli MM294 GFP biofilms was accomplished by using a Nikon A1+/AIR+ Confocal Microscope (Nikon Inc., Melville, NY, USA) with images acquired at 2 μm intervals through the biofilms. Two dimensional images were acquired using the Nis Elements Imaging software v. 4.20 (Nikon Inc., Melville, NY, USA). Experiments were done in triplicate.

### Assessing the Stability of the Selenium in the Tubing.

To access the stability of selenium-containing tubing, the selenium tubing was tested in the following manner. Tubing was polymerized with Se as described above, except that instead of drying after washing in PBS (phosphate buffered saline, pH 7.4), the selenium tubing was placed in glass tubes containing 10 mL of PBS. After incubation at 37° C. for 12 weeks, the tubing was sterilized by autoclaving. The tubing was then used in the biofilm assays described above to test for its ability to inhibit biofilm formation as described above. Statistical Analyses:

Results of the CFU assays were analyzed with GraphPad Prism® version 7 (GraphPad Software, San Diego, CA, USA) with 95% confidence intervals (CIs) of the difference. Comparisons of the in vitro biofilms formed on Se-free and Se-Polyurethane Tubings were analyzed, using GraphPad InStat 3 (GraphPad Software, San Diego, CA) with 95% confidence intervals (CIs) of the difference, by a two-tailed unpaired t-test to determine significant differences. In minimum, all experiments were done in triplicate. Differences were considered significant when the P-value was ≤0.05. Results were statistically analyzed using GraphPad InStat 3 (GraphPad Software, San Diego, CA) with 95% confidence intervals (CIs) of the difference.

The antibacterial study results are outlined in FIGS. 1-7. For both E. coli and K. pneumoniae, the bacteria grew over 7 logs in 24 hours on the control tubing, while none grew on the selenium tubing. Also, Haemophilus influenzae grew over 6 logs in 24 hours on the control tubing, but none grew on the selenium incorporate tubing of the present invention. Thus, the incorporation of selenium into the tubing material was effective in blocking the bacterial attachment and biofilm formation for each bacterial species.

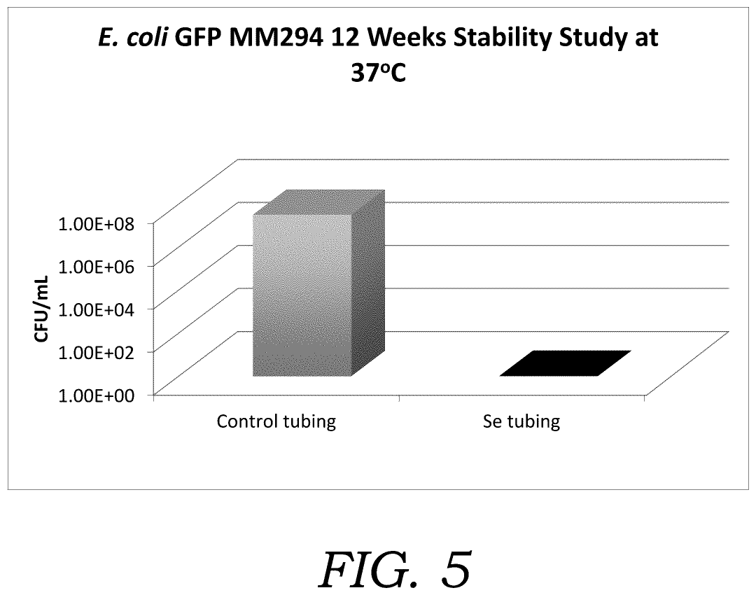

As mentioned previously, the stability of the selenium incorporated in the tubing was tested by soaking the tubing in PBS at 37° C. for 12 weeks. It was then treated with E. coli for 24 hours. As shown in FIG. 5, the untreated tubing had over 6 Logs of growth, while the selenium tubing had no growth.

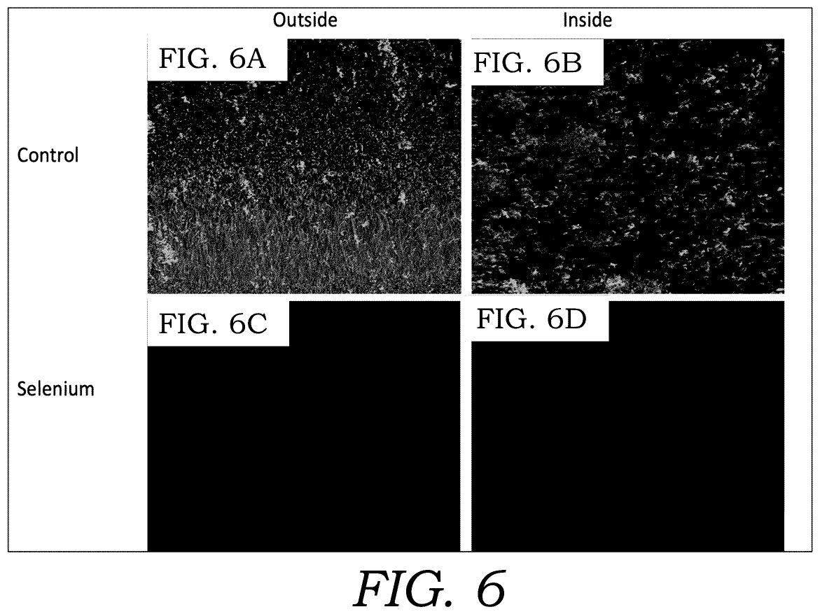

This same soaked technique was applied to tubing treated with the E. coli GFP MM294 and the test results were visualized with CLSM detecting the GFP protein in the E. coli as green fluorescence. As seen in FIG. 6, the E. coli grew on both the inside and on the outer surface of the control tubing, but no fluorescence was seen on the selenium tubing. FIG. 6A and FIG. 6B depict the outside and inside surfaces of the control tubing, respectively, while FIG. 6C and FIG. 6D depict the outside and inside surfaces of the selenium tubing, respectively.

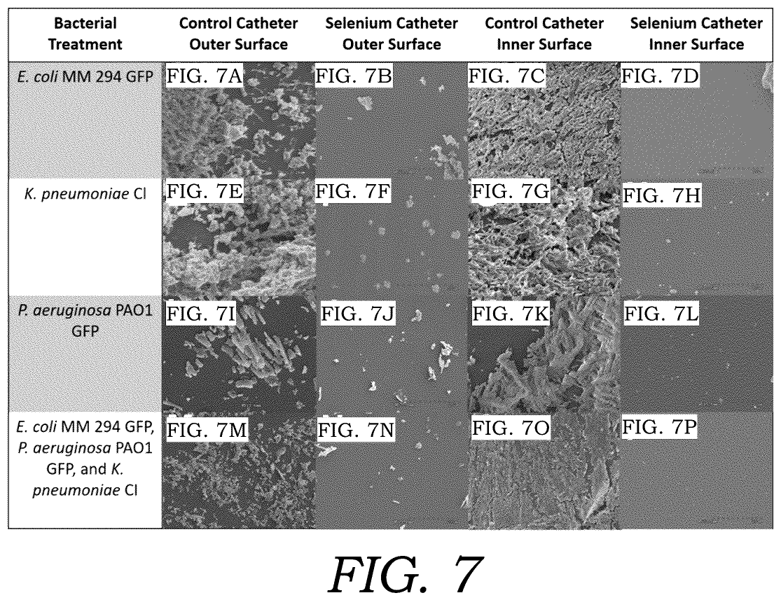

The 24-hour biofilms were also studied by Scanning Electron Microscopy (SEM) to visualize internal and external biofilm formation in control versus experimental catheters treated with E. coli, K. pneumoniae, P. aeruginosa, and all three pathogens combined, the results are summarized in FIG. 7. FIG. 7A, FIG. 7E, FIG. 7I, and FIG. 7M each depict the control catheter's outer surface treated with E. coli, K. pneumoniae, P. aeruginosa, and all three pathogens combined. FIG. 7B, FIG. 7F, FIG. 7J, and FIG. 7N each depict the selenium catheter's outer surface treated with E. coli, K. pneumoniae, P. aeruginosa, and all three pathogens combined. FIG. 7C, FIG. 7G, FIG. 7K, and FIG. 7O each depict the control catheter's outer surface treated with E. coli, K. pneumoniae, P. aeruginosa, and all three pathogens combined. FIG. 7D, FIG. 7H, FIG. 7L, and FIG. 7P each depict the selenium catheter's inner surface treated with E. coli, K. pneumoniae, P. aeruginosa, and all three pathogens combined. Clear differentiation is shown between the microbial activity of the control catheter and the selenium catheter based on the SEM images of the various surfaces.

The results of this study demonstrate that organo-selenium-coated catheters significantly inhibit the bacterial in-vitro growth and biofilm formation of E. coli, H. influenzae, and K. pneumoniae. Given that E. coli and K. pneumoniae represent the majority of commonly isolated bacteria in nosocomial UTIs [20], the substantial prevention of bacterial colonization on selenium tubing provides clinical pertinence in preventing CAUTIs. The addition of H. influenzae further demonstrates the ability of organo-selenium to inhibit unusual, less common CAUTI causative pathogens [21]. EM revealed qualitative growth inhibition of P. aeruginosa and multi-bacterial inoculation. P. aeruginosa is well known for its significant biofilm formation often very resistant to antibiotic therapy [22].

Prior non-urological studies have reported the effectiveness of covalent selenium in preventing bacterial colonization and biofilms against P. aeruginosa and S. aureus [13, 14]. These animal studies demonstrated that organo-selenium coatings are nontoxic to surrounding tissue [14], supporting the idea of implementing organo-selenium-coated catheters in future clinical trials. The antibacterial effectiveness of organo-selenium has been credited to its superoxide radicals that inhibit bacterial attachment on coated surfaces for a wide spectrum of pathogens [14,15].

CAUTIs are significant issues for patients and medical providers due to increased morbidity, mortality, and related costs. It is well documented that bacterial colonization and biofilm formation on urinary catheters cause persistent bacteriuria and antibacterial resistance, thus indicating the real need for preventative measures. Although other coated catheter technologies have demonstrated to be advantageous in preventing CAUTIs [12], organo-selenium has never been tested for this purpose. The literature supports the antibacterial value of organo-selenium and its nontoxicity to the surrounding tissue of the host. The results of this in-vitro study show that organo-selenium-incorporated catheters significantly inhibit the quantitative in-vitro growth and biofilm formation of E. coli, H. influenzae, and K. pneumoniae as well as the qualitative growth of P. aeruginosa and of combined bacterial species in comparison to control catheters. These results should encourage investigators to further evaluate its potential in the prevention of CAUTIs.

CAUTIs are common nosocomial infections contributing extensively to patient morbidity, mortality, and length of stay. Better prevention of CAUTIs would contribute to improved patient outcomes, lower healthcare costs, and reduced antibiotic-resistance. In the past antimicrobial coated catheters have proven to be effective in lowering the risks of CAUTIs. However, their clinical implementation has been limited by factors such as heterogeneity in antimicrobial substances, study design and limited statistical power, all factors which have prevented the wide clinical acceptance of antimicrobial catheter technologies.

The presented embodiments demonstrate that organo-selenium can be stably incorporated into catheter tubing to inhibit bacterial attachment, growth, and biofilm formation of multiple uropathogenic organisms. Polymerization of organo-selenium into catheter tubing material does not show any loss of antimicrobial activity even after soaking in PBS for 12 weeks at 37° C., thus demonstrating material stability and lasting effectiveness. The embodiments of the present invention show that organo-selenium-incorporated catheters may help in preventing catheter-associated urinary tract infections. This pilot study should encourage other investigators to proceed with in-vivo and randomized clinical trials in order to further establish the prophylactic values of organo-selenium hopefully achieving the long-term clinical goal of reducing bacteriuria and UTI episodes.

Those skilled in the art will recognize that the methods and compositions of the present invention may be implemented in many manners and as such are not to be limited by the foregoing exemplary embodiments and examples. Furthermore, the embodiments of methods presented and described in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

### REFERENCES

1. Singha P, Locklin J, Handa H. A review of the recent advances in antimicrobial coatings for urinary catheters. Acta biomaterialia. 2017; 50:20-40. https://dx.doi.org/10.1016/j.actbio.2016.11.070. doi: 10.1016/j.actbio.2016.11.070.
2. Toolkit for reducing catheter-associated urinary tract infections in hospital: Appendix D. poster on indications for urinary catheters. Agency for Healthcare Research and Quality, Rockville, M D Web site. https://www.ahrq.gov/hai/cauti-tools/impl-guide/implementation-guide-appendix-d.html. Updated 2015.
3. Nicolle L. Catheter associated urinary tract infections. Antimicrob Resist Infect Control. 2014; 3 (23). https://pubmed.ncbi.nlm.nih.gov/25075308/.
4. Lo J, Lange D, Chew B H. Ureteral stents and foley catheters-associated urinary tract infections: The role of coatings and materials in infection prevention. Antibiotics (Basel). 2014; 3 (1): 87-97. https://www.ncbi.nlm.nih.gov/pubmed/27025736. doi: 10.3390/antibiotics3010087.
5. Chant C, Smith O M, Marshall J C, Friedrich J O. Relationship of catheter-associated urinary tract infection to mortality and length of stay in critically ill patients: A systematic review and meta-analysis of observational studies. Critical care medicine. 2011; 39 (5): 1167-1173. http://ovidsp.ovid.com/ovidweb.cgi?T=JS&NEWS=n&CSC=Y&PAGE-fulltext&D=ovft&AN=00003246-201105000-00034. doi: 10.1097/CCM.0b013e31820a8581.
6. Majumder M M I, Ahmed T, Ahmed S, Khan A R. Microbiology of catheter associated urinary tract infection. In: Behzadi P, ed. Microbiology of urinary tract infections-microbial agents and predisposing factors. IntechOpen; 2018. https://www.intechopen.com/chapters/62973. doi: 10.5772/intechopen.80080.
7. Peng D, Li X, Liu P, et al. Epidemiology of pathogens and antimicrobial resistance of catheter-associated urinary tract infections in intensive care units: A systematic review and meta-analysis. American journal of infection control. 2018; 46 (12): e81-e90. https://dx.doi.org/10.1016/j.ajic.2018.07.012. doi: 10.1016/j.ajic.2018.07.012.
8. Weiner L M, Webb A K, Limbago B, et al. Antimicrobial-Resistant Pathogens Associated With Healthcare-Associated Infections: Summary of Data Reported to the National Healthcare Safety Network at the Centers for Disease Control and Prevention, 2011-2014. Infect Control Hosp Epidemiol. 2016; 37 (11): 1288-1301. doi: 10.1017/ice.2016.174.
9. Karchmer T B, Giannetta E T, Muto C A, Strain B A, Farr B M. A randomized crossover study of silver-coated urinary catheters in hospitalized patients. Archives of internal medicine (1960). 2000; 160 (21): 3294-3298. http://dx.doi.org/10.1001/archinte.160.21.3294. doi: 10.1001/archinte. 160.21.3294.
10. Stenzelius K, Persson S, Olsson U, Stjärneblad M. Noble metal alloy-coated latex versus silicone foley catheter in short-term catheterization: A randomized controlled study. Scandinavian journal of urology and nephrology. 2011; 45 (4): 258-264. http://www.tandfonline.com/doi/abs/10.3109/00365599.2011.560007. doi: 10.3109/00365599.2011.560007.
11. STENSBALLE J, TVEDE M, LOOMS D, et al. Infection risk with nitrofurazone-impregnated urinary catheters in trauma patients: A randomized trial. Annals of internal medicine. 2007; 147 (5): 285-293. https://www.ncbi.nlm.nih.gov/pubmed/17785483. doi: 10.7326/0003-4819-147-5-200709040-00002.
12. Vopni R, Voice A, de Riese C S, Garza J, de Riese W T. Use of antimicrobial-coated catheters in preventing catheter-associated urinary tract infections and bacteriuria: A meta-analysis for clinicians. Urology Practice. 2021; 8 (6): 705-712. doi: 10.1097/UPJ.0000000000000254.
13. Vatansever F, de Melo W C, Avci P, et al. Antimicrobial strategies centered around reactive oxygen species—bactericidal antibiotics, photodynamic therapy, and beyond. FEMS Microbiol Rev. 2013; 37 (6): 955-989. doi: 10.1111/1574-6976.12026
14. Mathews S, Spallholz J, Grimson M, Dubielzig R, Gray T, Reid T. Prevention of bacterial colonization of contact lenses with covalently attached selenium and effects on the rabbit cornea. Cornea. 2006; 25 (7): 806-814. http://ovidsp.ovid.com/ovidweb.cgi?T=JS&NEWS-n&CSC=Y&PAGE=fulltext&D=o vft&AN=00003226-200608000-00009. doi: 10.1097/01.ico.0000224636.57062.90.
15. TRAN P L, LOWRY N, HAMOOD A N, et al. An organoselenium compound inhibits Staphylococcus aureus biofilms on hemodialysis catheters in vivo. Antimicrobial Agents and Chemotherapy. 2012; 56 (2): 972-978. http://aac.asm.org/content/56/2/972.abstract. doi: 10.1128/AAC.05680-11.
16. Narayanan A, Nair M S, Muyyarikkandy M S, Amalaradjou M A. Inhibition and inactivation of uropathogenic Escherichia coli biofilms on urinary catheters by sodium selenite. International journal of molecular sciences. 2018; 19 (6): 1703. https://www.ncbi.nlm.nih.gov/pubmed/29880781. doi: 10.3390/ijms19061703.
17. Phat L Tran, Eric Huynh, Abdul N Hamood, Anselm de Souza, Gregory Schultz, Bernd Liesenfeld, Dilip Mehta, Daniel Webster, Ted W Reid, The ability of quaternary ammonium groups attached to a urethane bandage to inhibit bacterial attachment and biofilm formation in a mouse wound model, International Wound Journal, 10.1111/iwj.12554, 14, 1, (79-84), (2015).
20. JACOBSEN S M, STICKLER D J, MOBLEY H L T, SHIRTLIFF M E. Complicated catheter-associated urinary tract infections due to Escherichia coli and Proteus mirabilis. Clinical Microbiology Reviews. 2008; 21 (1): 26-59. http://cmr.asm.org/content/21/1/26.abstract. doi: 10.1128/CMR.00019-07.
21. Diedrich L K, Manby C L. Haemophilus species as a urinary tract pathogen. Laboratory medicine. 2017; 48 (1): e1-e3. https://www.ncbi.nlm.nih.gov/pubmed/28039378. doi: 10.1093/labmed/lmw063.
22. Cole S J, Records A R, Orr M W, Linden S B, Lee V T. Catheter-associated urinary tract infection by Pseudomonas aeruginosa is mediated by exopolysaccharide-independent biofilms. Infection and immunity. 2014; 82 (5): 2048-2058. https://www.ncbi.nlm.nih.gov/pubmed/24595142. doi: 10.1128/IAI.01652-14.
23. P. L. Tran, A. A. Hammond, T. Mosley, J. Cortez, T. Gray, J. A. Colmer-Hamood, et al. Organoselenium coating on cellulose inhibits the formation of biofilms by Pseudomonas aeruginosa and Staphylococcus aureus Appl Environ Microbiol, 75 (2009), pp. 3586-3592
24. W. J. Brown National Committee for Clinical Laboratory Standards agar dilution susceptibility testing of anaerobic gram-negative bacteria Antimicrob Agents Chemother, 32 (1988), pp. 385-390

1. An anti-microbial catheter tube, wherein the catheter tube comprises:
(a) an outer surface; and
(b) an inner surface positioned in the interior of the outer surface,
wherein:
said inner surface comprises a polymer/plastic,
said inner surface is operable for microbial contact with the surface,
said surface comprises an organo-selenium composition incorporated into the polymer/plastic, and
said organo-selenium composition is operable to generate superoxide free radical species upon contact with microbes to inhibit microbial growth on said inner surface, and said organo-selenium composition does not leach from said inner surface.

2. The catheter tube of claim 1, wherein:
said outer surface comprises a polymer/plastic,
said outer surface is operable for microbial contact with the surface,
said surface comprises an organo-selenium composition incorporated into the polymer/plastic, and
said organo-selenium composition is operable to generate superoxide free radical species upon contact with microbes to inhibit microbial growth on said outer surface, and said organo-selenium composition does not leach from said outer surface.

3. The catheter tube of claim 1, wherein said organo-selenium composition is fixed within the polymer/plastic.

4. The catheter tube of claim 1, wherein said organo-selenium composition is incapable of escaping the polymer/plastic.

5. The catheter tube of claim 1, wherein said polymer/plastic is selected from a group consisting of: nylon, declon, polyvinyl chloride, Polyvinyl chloride, silicone, poly butadiene, polyacrylonitrile, methacrylates, polyactic acid, aromatic polyesters thermoplastic polyurethane resin, high density polyethylene, high pressure, low density polyethylene, polypropylene, high-impact polystyrene, polyamides, PVC plasticisers, ABS, MBS, and combinations thereof.

6. The catheter tube of claim 1, wherein said organo-selenium composition comprises a diselenide.

7. The catheter tube of claim 1, wherein said organo-selenium composition is selected from a group consisting of: diselenide (—Se—Se—), selenol (—SeH), diselenide methacrylate, compounds capable of polymerization having a selenol, compounds capable of polymerization having a diselenide, and combinations thereof.

8. The catheter tube of claim 1, wherein said organo-selenium composition is incorporated into the polymer/plastic as a coating on one or more layers of polymer/plastic film.

9. The catheter tube of claim 1, wherein said organo-selenium composition is distributed throughout said polymer/plastic.

10. The catheter tube of claim 9, wherein said organo-selenium composition is uniformly distributed throughout said polymer/plastic.

11. A method for inhibiting microbial growth on a catheter tube, comprising: selecting a catheter tube comprising an organo-selenium composition, wherein said organo-selenium composition diminishes or inhibits microbial growth on the surface of said catheter tube.

12. The method of claim 11, wherein said organo-selenium composition is fixed within the polymer/plastic.

13. The method of claim 11, wherein the polymer/plastic is selected from a group consisting of: nylon, declon, polyvinyl chloride, Polyvinyl chloride, silicone, poly butadiene, polyacrylonitrile, methacrylates, polyactic acid, aromatic polyesters thermoplastic polyurethane resin, high density polyethylene, high pressure, low density polyethylene, polypropylene, high-impact polystyrene, polyamides, PVC plasticisers, ABS, MBS, and combinations thereof.

14. The method of claim 11, wherein the organo-selenium composition comprises a diselenide.

15. The method of claim 11, wherein the organo-selenium composition is selected from a group consisting of diselenide (—Se—Se—), selenol (—SeH), diselenide methacrylate, compounds capable of polymerization having a selenol, compounds capable of polymerization having a diselenide, and combinations thereof.

16. The method of claim 11, wherein said inhibition of microbial growth occurs through contact with said surface of said catheter tubing.

17. The method of claim 11, wherein said inhibition of microbial growth includes inhibition of the formation biofilms.

18. A method for making a catheter tube capable of microbial inhibition, wherein the method comprises forming a catheter tube having a surface that comprises a organo-selenium composition incorporated into the polymer/plastic, wherein incorporated said organo-selenium composition is operable to inhibit microbial species present on the surface of said catheter tube such that microbial growth or biofilm formation is diminished or prevented, and said organo-selenium composition does not leach from said inner surface.

19. The method of claim 18, wherein said polymer/plastic is selected from a group consisting of nylon, declon, polyvinyl chloride, Polyvinyl chloride, silicone, poly butadiene, polyacrylonitrile, methacrylates, polyactic acid, aromatic polyesters thermoplastic polyurethane resin, high density polyethylene, high pressure, low density polyethylene, polypropylene, high-impact polystyrene, polyamides, PVC plasticisers, ABS, MBS, and combinations thereof.
